(19) United States
(12) Patent Application Publication
(10) Pub. No.: US 2026/0083434 A1
(43) Pub. Date: Mar. 26, 2026

(54) ULTRASOUND PROBE WITH IMPROVED DROP ROBUSTNESS AND HIGH SCANNING VOLUME

(71) Applicant: GE Precision Healthcare LLC, Waukesha, WI (US)

(72) Inventors: Christian Heinrich, Neukirchen (AT); Krystof Jezek, Vocklabruck (AT); Bernhard Aschenbrenner, Altmuenster am Traunsee (AT); Reinhard Wimmer, Aurolzmuenster (AT); Wolfgang Knoll, Zipf (AT)

(73) Assignee: GE Precision Healthcare LLC, Waukesha, WI (US)

(21) Appl. No.: 19/333,031

(22) Filed: Sep. 18, 2025

Related U.S. Application Data
(60) Provisional application No. 63/698,016, filed on Sep. 23, 2024.

(51) Int. Cl. A61B 8/00; A61B 8/08

(52) U.S. Cl. CPC A61B 8/4461; A61B 8/08; A61B 8/4405; A61B 8/4494; A61B 8/461

(57) ABSTRACT

Systems and methods for providing an ultrasound probe with improved drop robustness and high scanning volume are described herein. In one example, an ultrasound imaging system includes a probe. The probe includes a distal portion, a proximal portion, and a drive mechanism. The proximal portion includes a transducer configured to transmit and receive an ultrasound signal, a bearing configured to facilitate rotational movement of the transducer, and a pulley system. The pulley system includes a pulley and a cord having an intersection point. The drive mechanism is configured to impart a force on the transducer via the pulley such that the force is applied at an angle defined by the intersection point of the cord.

### CROSS-REFERENCE TO RELATED PATENT APPLICATIONS

This application claims the benefit of and priority to U.S. Provisional Patent Application No. 63/698,016, filed Sep. 23, 2024, which is incorporated herein by reference in its entirety and for all purposes.

### FIELD

Embodiments of the subject matter disclosed herein relate to ultrasound imaging, and more particularly, to an ultrasound probe having improved drop robustness and high scanning volume.

### BACKGROUND

During a medical imaging scan, a plurality of medical images of a patient are obtained by a technician, such as a sonographer, to measure or detect various aspects of anatomical features present within the medical images. In some instances, the plurality of medical images are obtained by inserting a probe (e.g., an endocavity probe) into an anatomical cavity (e.g., a vagina, a rectum) to obtain high-resolution images of nearby internal structures for diagnostic evaluation.

### SUMMARY

An embodiment relates to an ultrasound imaging system. The ultrasound imaging system includes a probe. The probe includes a distal portion, a proximal portion, and a drive mechanism. The proximal portion includes a transducer configured to transmit and receive an ultrasound signal, a bearing configured to facilitate rotational movement of the transducer, and a pulley system. The pulley system includes a pulley and a cord having an intersection point. The drive mechanism is configured to impart a force on the transducer via the pulley such that the force is applied at an angle defined by the intersection point of the cord.

Another embodiment relates to an ultrasound probe. The ultrasound probe includes a distal portion, a proximal portion, and at least one drive mechanism. The proximal portion includes a transducer configured to transmit and receive an ultrasound signal, a bearing configured to facilitate rotational movement of the transducer, and a pulley system. The pulley system includes a pulley and a cord having an intersection point. The drive mechanism is configured to impart a force on the transducer via the at least one pulley such that the force is applied at an angle defined by the intersection point of the cord.

Another embodiment relates to an ultrasound imaging system. The ultrasound imaging system includes a probe. The probe includes a distal portion, a proximal portion, a seal disposed between the distal portion and the proximal portion, and a drive mechanism. The proximal portion includes a transducer configured to transmit and receive an ultrasound signal, a bearing configured to facilitate rotational movement of the transducer, and a pulley system. The pulley system includes a pulley and a cord having an intersection point. The seal is configured to prevent an oil volume within the proximal portion from reaching the distal portion. The drive mechanism is configured to impart a force on the transducer via the at least one pulley such that the force is applied at an angle defined by the intersection point of the cord.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

FIG. 1 is an illustration of an ultrasound imaging system, according to an example embodiment.

FIG. 2 is a block diagram of the ultrasound imaging system of FIG. 1, according to an example embodiment.

FIG. 3 is a block diagram of a processing circuit of the ultrasound imaging system of FIG. 2, according to an example embodiment.

FIG. 4 is a flow chart illustrating a method for performing an ultrasound imaging procedure, according to an example embodiment.

FIG. 5 is an illustration of a probe of the ultrasound imaging system of FIG. 1, according to an example embodiment.

FIG. 6A is a first illustration of internal mechanics of the probe of FIG. 5, according to an example embodiment.

FIG. 6B is a second illustration of the internal mechanics of the probe of FIG. 5, according to an example embodiment.

FIG. 6C is a third illustration of the internal mechanics of the probe of FIG. 5, according to an example embodiment.

FIG. 7A is a first illustration of external features of the probe of FIG. 5, according to an example embodiment.

FIG. 7B is a second illustration of the external features of the probe of FIG. 5, according to an example embodiment.

FIG. 8 is a flow chart illustrating a method for using the probe of FIG. 5 during an ultrasound procedure, according to an example embodiment.

### DETAILED DESCRIPTION

Referring generally to the figures, systems and methods for an improved ultrasound probe having drop robustness and high scanning volume are disclosed. More specifically, the systems and methods described herein include configuring mechanics of the ultrasound probe within a tip of the ultrasound probe such that an oil volume of the probe is reduced, rotational movement of the probe is increased, and the field of view of the transducer improved.

In existing systems, mechanical features (e.g., a pulley mechanism) of an ultrasound probe are located within a distal portion of the ultrasound probe (e.g., the handle). In such configurations, an oil volume within the probe extends from a tip of probe through the handle such that ultrasound waves may propagate to and from the transducer across a gap between the transducer and housing. Further, in such configurations, an oil volume within the probe extends from a tip of probe through the handle such that the mechanical features disposed therein (e.g., the pulley mechanism) can operate. Furthermore, existing systems include a bearing disposed around an external surface of the transducer, which limits the field of view obtainable by the transducer.

The systems and methods described herein, however, provide a technical solution to existing systems by improving the configuration of an ultrasound probe by housing the mechanics within a tip of the ultrasound probe. That is, by housing mechanical features of the ultrasound probe within a tip (e.g., proximal portion, proximal end, etc.) of the probe, the oil volume included in the probe may be reduced, making the probe lighter in weight and thereby improving reliability, facilitating handling, and reducing an impact force of the probe. Furthermore, having a reduced oil volume compared to existing ultrasound probes causes the ultrasound probe to be more drop resistant, which decreases the likelihood of damage to the probe. As yet another technical solution, the systems and methods described herein provide a larger scan volume achievable by the transducer, allowing the ultrasound probe to capture more areas of interest compared to existing ultrasound probes. In this way, scanning time during a procedure may be reduced because the increased scan volume of the transducer allows the system to reach areas of interest in less time. Further, the larger scan volume allows the system to reach areas originally unreachable with lower scan volume (e.g., limited movement possibilities. For example, the larger scan volume may capture an entire region of interest (ROI) in a singular image, while the lower scan volume may capture the entire ROI in several images by moving the probe to visualize the ROI.

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

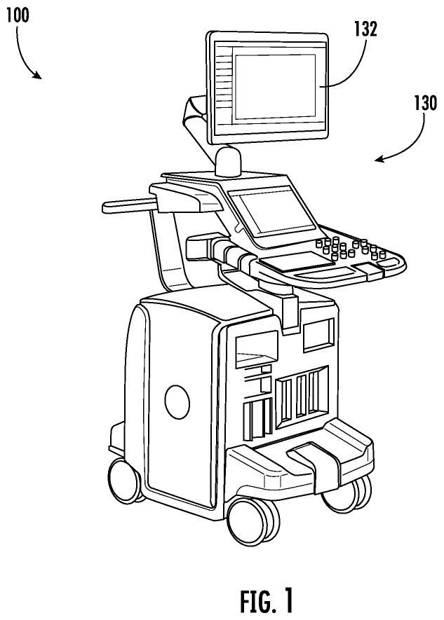

Referring to FIG. 1, an ultrasound imaging system 100 is shown. Specifically, the ultrasound imaging system 100 is shown to include a user interface 130 and a display device 132. The ultrasound imaging system 100 may be used in a medical environment (e.g., hospitals, clinics, etc.), for example, by a sonographer, technician, or other clinician certified to collect ultrasound data from a patient. Although the systems and methods are described herein in the context of the ultrasound imaging system 100, it should be appreciated that the improved probe configuration described herein may be implemented using any of a variety of medical imaging systems (e.g., medical resonance imaging, x-ray, computed tomography, positron emission tomography, etc.).

The user interface 130 may be used by a sonographer or other clinician to control operation of the ultrasound imaging system 100. For example, the sonographer may use the user interface 130 to control the input of patient data, to change a scanning or display parameter, to adjust a segmentation of an anatomical feature depicted in an ultrasound image, and/or to select various other modes, operations, parameters, etc. of the ultrasound imaging system 100. In some embodiments, the user interface 130 may include an off-the-shelf consumer electronic device such as a smartphone, a tablet, a laptop, and so on. For the purposes of this disclosure, the term “off-the-shelf consumer electronic device” is defined to be an electronic device that was designed and developed for general consumer use and one that was not specifically designed for use in a medical environment. Alternatively, in other embodiments, the user interface 130 may be an electronic device that was designed and developed for use in a medical environment.

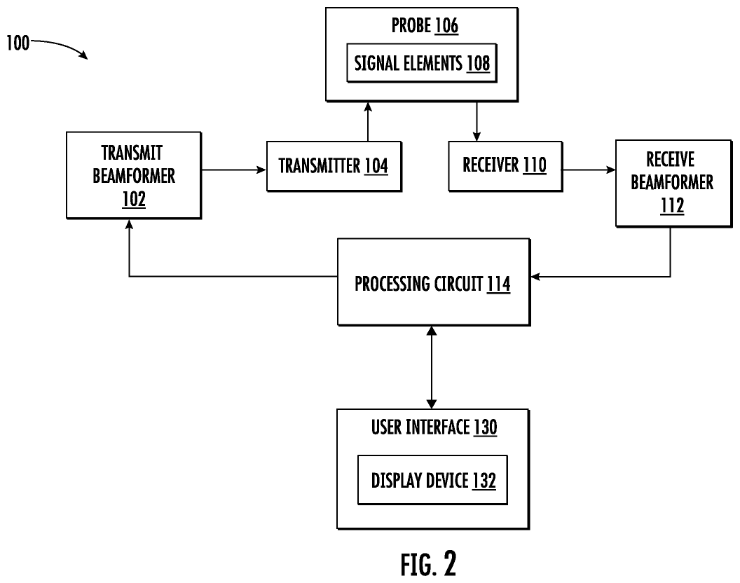

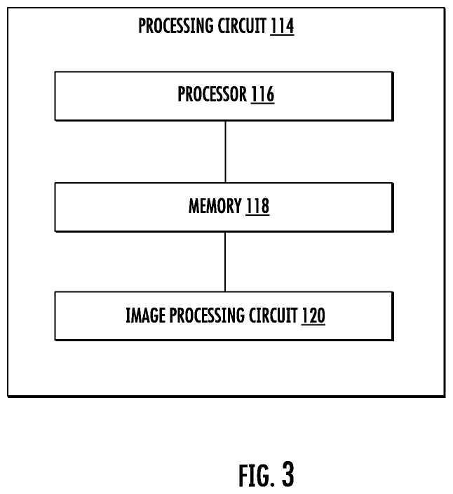

According to some embodiments, the user interface 130 may be physically separate from the rest of the ultrasound imaging system 100 (e.g., the transmit beamformer 102, the transmitter 104, the probe 106, the receiver 110, the receive beamformer 112, and/or the processing circuit 114, as shown in FIG. 2). The user interface 130 may communicate with a processor (e.g., processor 116 as shown in FIG. 3) through a wireless protocol, such as Wi-Fi, Bluetooth, wireless local area network (WLAN), near-field communication, and so on. According to some embodiments, the user interface 130 may communicate with the processor through an application programming interface (API). In some embodiments, the user interface 130 may include physical controls such as one or more of buttons, sliders, a rotary knob, a mouse, a keyboard, a trackball, hard keys linked to specific actions, soft keys that may be configured to control different functions, and so on.

As shown in FIG. 1, the ultrasound imaging system 100 may also include a display device 132. In some embodiments, as shown in FIG. 2, the display device 132 may be part of the user interface 130. The display device 132 may be a screen configured to display a graphical user interface (GUI) based on an instruction from a memory (e.g., memory 118, as shown in FIG. 3). The GUI may include user interface icons representing commands and instructions relating to the operation of the ultrasound imaging system 100. The user interface icons of the GUI may be configured such that a user (e.g., the sonographer, clinician, etc.) may select a specific user interface icon in order to initiate a specific function controlled by the GUI. For example, various user interface icons may be used to represent windows, menus, buttons, cursors, scroll bars, and so on. That is, the physical controls of the user interface 130 may be included as individual hardware elements, as user interface icons displayed on the display device 132, or as a combination of hardware elements and user interface icons.

In some embodiments, the display device 132 may include a touch-sensitive display device or a touch screen. According to such embodiments, the touch screen may be configured to interact with the GUI displayed by the display device 132 such that a user (e.g., the sonographer) can interact with the GUI via the touch screen. The touch screen may be a single-point touch screen that is configured to detect a single contact point at a time, or the touch screen may be a multi-point touch screen that is configured to detect multiple points of contact at a time. For embodiments where the touch screen is a multi-point touch screen, the touch screen may be configured to detect multi-point gestures involving contact from two or more of a user's fingers at a time. The touch screen may be a resistive touch screen, a capacitive touch screen, or any other type of touch screen that is configured to receive inputs from a stylus or one or more of a user's fingers. According to some embodiments, the touch screen may be an optical touch screen that uses technology such as infrared light or other frequencies of light to detect one or more points of contact initiated by a user. In some embodiments, the touch screen may be incorporated as part of the display device 132 or may be separate from the display device 132.

The user interface 130 may also include a proximity sensor configured to detect objects and/or gestures that are within a predetermined distance (e.g., five feet, six inches, ten centimeters, etc.) of the proximity sensor. In various embodiments, the proximity sensor may be located on the display device 132 or as part of a touch screen that is separate from the display device 132.

Referring to FIG. 2, a block diagram of the ultrasound imaging system 100 is shown. As shown in FIG. 3, the ultrasound imaging system 100 includes a transmit beamformer 102, a transmitter 104, a probe 106, a receiver 110, and a receive beamformer 112.

The transmit beamformer 102 may be either a hardware beamformer or a software beamformer. In embodiments where the transmit beamformer 102 is a hardware beamformer, the transmit beamformer 102 may include one or more of a graphics processing unit (GPU), a microprocessor, a central processing unit (CPU), a digital signal processor (DSP), or any other type of processor capable of performing logical operations. The transmit beamformer 102 may be configured to perform conventional beamforming techniques as well as techniques such as retrospective transmit beamforming (RTB). Alternatively, in embodiments where the transmit beamformer 102 is a software beamformer, a processor (e.g., processor 116, as described below) may be configured to perform some or all of the functions associated with the transmit beamformer 102.

The probe 106 may be a linear array probe, a curvilinear array probe, a sector probe, or any other type of probe configured to obtain two-dimensional (2D) B-mode data, 2D color flow data, M-mode data, three-dimensional (3D) data, four-dimensional (4D) data, or any other type of ultrasound data. Alternatively or additionally, the probe 106 may be any type of probe configured to obtain 2D B-mode data and data corresponding to another ultrasound mode that detects blood flow velocity in the direction of a vessel axis. In some embodiments, the probe 106 may include a position sensor configured to detect a position of the probe 106 relative to one or more reference locations. That is, the position sensor may continuously track movement (e.g., rotation, translation, orientation, etc.) of the probe 106 relative to the location of the probe 106 when the anatomy being imaged is identified. For example, the anatomy being imaged may be identified as a uterus at a first location of the probe 106. Then, the position sensor may track the movement of the probe 106 relative to the uterus in order to identify successive locations of the probe 106. In some embodiments, the position sensor may transmit position data to be stored within the ultrasound imaging system 100 (e.g., in memory 118, as shown in FIG. 3).

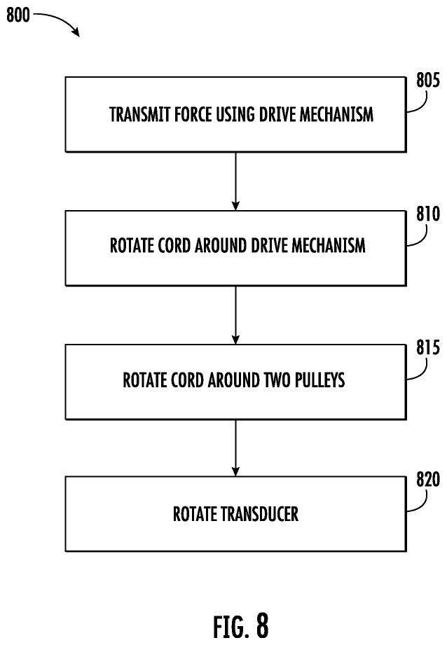

In some embodiments, the probe 106 may be an endocavity probe (e.g., an endocavitary transducer). An endocavity ultrasound probe refers to a probe configured to perform internal imaging procedures through body cavities (e.g., the vagina, the rectum, etc.). The endocavity probe may comprise a compact, elongated shape compared to an external (e.g., linear, curvilinear, etc.) probe. In this way, during an ultrasound imaging procedure, the endocavity probe may be configured to achieve proximity to internal organs (e.g., the uterus, ovaries, prostate, or rectal wall). By emitting high-frequency sound waves, the endocavity probe captures ultrasound images of such internal organs that may be used in diagnosing conditions such as pelvic abnormalities, early pregnancies, or prostate disorders. Endocavity probes are commonly used in gynecological, obstetric, and urological examinations, offering enhanced image clarity compared to external ultrasound techniques (e.g., using a linear probe, a curvilinear probe, etc.). An example of an ultrasound imaging procedure using an endocavity probe is described in greater detail below, with reference to FIG. 8.

The probe 106 may include a transducer configured to transmit and receive an ultrasound signal. In some embodiments, as shown in FIG. 2, the probe 106 includes signal elements 108. The signal elements 108 may be arranged in a transducer array, and in some embodiments may be arranged in a one-dimensional (1D) or 2D array. The transmit beamformer 102 and the transmitter 104 drive the signal elements 108 to emit pulsed ultrasonic signals into a body of a subject (e.g., a patient). For example, during a gynecological examination, a sonographer or other clinician may navigate the probe 106 proximate to a patient's uterus so that the signal elements 108 in the probe 106 emit the pulsed ultrasonic signals into the patient's uterus. The pulsed ultrasonic signals are then back-scattered from anatomical structures in the body, such as blood cells or muscular tissues, to produce echoes that return to the signal elements 108. That is, the signal elements 108 may include the transducer configured to transmit and receive the ultrasound signal, a matching layer configured to have an acoustic impedance match between a tissue to be imaged and a material of the transducer (e.g., such that the pulsed electronic signals can be back-scattered from the anatomical structures in the body and received as echoes by the signal elements 108), and a damping block configured to absorb ultrasound energy.

The receiver 110 receives the echoes from the probe 106 and converts the echoes into electrical signals. The electrical signals are then passed through the receive beamformer 112, which produces the ultrasound data from the electrical signals. As described above with reference to the transmit beamformer 102, the receive beamformer 112 may be either a hardware beamformer or a software beamformer. In embodiments where the receive beamformer 112 is a hardware beamformer, the receive beamformer 112 may include one or more of a GPU, a microprocessor, a CPU, a DSP, or any other type of processor capable of performing logical operations. The receive beamformer 112 may be configured to perform conventional beamforming techniques as well as techniques such as retrospective transmit beamforming (RTB). Alternatively, in embodiments where the receive beamformer 112 is a software beamformer, a processor (e.g., processor 116, as shown in FIG. 3) may be configured to perform some or all of the functions associated with the receive beamformer 112.

Although the transmit beamformer 102, the transmitter 104, the receiver 110, and the receive beamformer 112 are shown in FIG. 2 as being components of the ultrasound imaging system 100 that are distinct from the probe 106, it should be appreciated that in some embodiments, the probe 106 may include electronic circuitry configured to perform the functions of each of the transmit beamformer 102, the transmitter 104, the receiver 110, and/or the receive beamformer 112. That is, all or part of the transmit beamformer 102, the transmitter 104, the receiver 110, and/or the receive beamformer 112 may be situated within the probe 106.

Referring still to FIG. 2, the ultrasound imaging system 100 is shown to include a processing circuit 114. While shown as being separate from the probe 106 in FIG. 2, it will be appreciated that the processing circuit 114 can be part of the probe 106. For example, the processing circuit 114 can be disposed in a handheld housing of the probe 106 (e.g., in the case of the probe 106 being a wireless probe).

The ultrasound imaging system 100 is also shown to include the user interface 130 and the display device 132, as described above in greater detail above with reference to FIG. 1.

Referring to FIG. 3, the processing circuit 114 of the ultrasound imaging system 100 is shown in greater detail. As shown, the processing circuit 114 may include at least one processor 116, a memory 118, and an image processing circuit 120. In this way, the processing circuit 114 may be structured or configured to execute or implement the instructions, commands, and/or control processes described herein with respect to the processor 116, the memory 118, and the image processing circuit 120.

The processor 116 may include a CPU, a GPU, a microprocessor, a DSP, a general-purpose single- or multi-chip processor, a field-programmable gate array (FPGA), or any other type of processor capable of performing logical operations. A general-purpose processor may be a microprocessor, or, any conventional processor, or state machine. A processor also may be implemented as a combination of computing devices, such as a combination of a DSP and a microprocessor, a plurality of microprocessors, one or more microprocessors in conjunction with a DSP core, or any other such configuration. In some embodiments, the processor 116 may be shared by multiple circuits (e.g., the circuits of the processor 116 may include or otherwise share the same processor which, in some example embodiments, may execute instructions stored, or otherwise accessed, via different areas of the memory 118). Alternatively or additionally, the processor 116 may be structured to perform or otherwise execute certain operations independent of one or more co-processors. In some embodiments, two or more processors may be coupled via a bus to enable independent, parallel, pipelined, or multi-threaded instruction execution. All such variations are intended to fall within the scope of the present disclosure.

The processor 116 may be configured to control the transmit beamformer 102, the transmitter 104, the receiver 110, and the receive beamformer 112. The processor 116 may also be in electronic communication with the probe 106. For purposes of this disclosure, the term “electronic communication” may be defined to include both wired and wireless communications.

In some embodiments, the processor 116 may be configured to control the probe 106 during data acquisition. That is, the processor 116 may control the data acquisition by controlling which of the signal elements 108 are active and by controlling a shape of the beam emitted from the probe 106. Alternatively or additionally, the processor 116 may include a complex demodulator configured to demodulate radio frequency (RF) data obtained by the probe 106 and generate raw data. According to other embodiments, the demodulation of the RF data may be performed by another component of the ultrasound imaging system 100. The processor 116 may perform the processing operations described herein according to a plurality of selectable ultrasound modalities.

Depending on a mode of operation of the ultrasound imaging system 100, the processor 116 may process ultrasound data obtained by the probe 106 according to the mode of operation to generate 2D or 3D image data. For example, the mode of operation may include B-mode, color flow Doppler mode, M-mode, color M-mode, spectral Doppler, elastography, TVI, strain, strain rate, and the like. Various of these modes of operation may be configured to, for instance, convert ultrasound data from beam space coordinates (e.g., received from the receive beamformer 112) to display space coordinates (e.g., such that the ultrasound data may be displayed as image data). In some embodiments, the mode of operation may allow for video processing by the processor 116 such that a series of images (e.g., processed ultrasound data) may be displayed in real-time while a scanning session/procedure is being performed on a patient. An operator of the ultrasound imaging system 100 (e.g., a sonographer) may switch between various modes in order to obtain a variety of ultrasound data and to perform a complete scan of an anatomical region of interest. For example, the operator may switch between modes using user interface 130 (e.g., using physical controls, interface inputs representing physical controls, etc.). While the term “image” or “images” are used herein to for the purposes of example, it will be appreciated that such terms cover still images as well as videos, clips, or a series of images for each. For example, in some embodiments, the image or images may include a 1-2 second clip derived from the image data.

The processor 116 performs the processing operations in real-time as the echo signals are received by the receiver 110 from the probe 106. For the purposes of this disclosure, the term “real-time” is defined to include a procedure that is performed without any intentional delay. As an illustrative, non-limiting example, in certain instances, the ultrasound imaging system 100 may obtain images at a real-time volume-rate of 7-20 volumes/sec. It should be appreciated, however, that the real-time volume-rate may be dependent on the length of time that it takes to obtain each volume of data for display. Thus, the ultrasound imaging system 100 may be configured to obtain 2D data of an anatomical region at a faster rate than 3D data of the same anatomical region because it takes longer to obtain a volume of 3D data than the same volume of 2D data. Similarly, when the ultrasound imaging system 100 obtains a relatively large volume of data, the real-time volume-rate may be slower than for a smaller volume of data. For example, during an abdominal scan, the real-time volume-rate may be slower if the patient is an adult versus if the patient is an infant because the volume of data is larger for the adult than for the infant (e.g., due to the abdomen of an adult being larger than the abdomen of an infant). Therefore, certain implementations of the ultrasound imaging system 100 may have real-time volume-rates that are faster than 20 volumes/sec, while other implementations of the ultrasound imaging system 100 may have real-time volume-rates that are slower than 7 volumes/sec.

In some embodiments, the ultrasound imaging system 100 may include multiple processors configured to perform the processing operations/functionality described with reference to processor 116. For example, in such embodiments, a first processor of the multiple processors may be configured to demodulate and decimate the RF signal while a second processor of the multiple processors may be configured to further process the RF data prior to displaying an image representative of the data. It should be appreciated that other embodiments may use a different arrangement of processors.

The processor 116 may also be in electronic communication with the display device 132 such that the processor 116 may process ultrasound data obtained by the probe 106 and generate images to display on the display device 132.

As shown in FIG. 3, the processing circuit 114 also includes the memory 118. The memory 118 may be configured to, for example, store processed volumes of data obtained by the ultrasound imaging system 100 (e.g., ultrasound data collected by the probe 106, user inputs received by the user interface 130, etc.). For example, the memory 118 may be a hospital picture archiving and communication system (PACS). The memory 118 (e.g., memory, memory unit, storage device, etc.) may include one or more devices (e.g., RAM, ROM, Flash memory, hard disk storage, etc.) for storing data and/or computer code for completing or facilitating the processes, layers, and modules described in the present application. The memory 118 may be or include tangible, non-transient volatile memory or non-volatile memory. The memory 118 may also include database components, object code components, script components, or any other type of information structure for supporting the activities and information structures described in the present application.

In various embodiments, the memory 118 may have varying capacity (e.g., storage space) across embodiments of the ultrasound imaging system 100. For example, the memory 118 may be configured to store at least 60 minutes' worth of ultrasound data. The ultrasound data may be stored in the memory 118 such that the ultrasound data may be retrieved according to an order/time of acquiring the data. That is, the ultrasound data may be stored with a timestamp indicating a time at which the ultrasound data was collected and may be retrieved starting with an oldest time at which the ultrasound data was collected.

The processing circuit 114 also includes the image processing circuit 120. The image processing circuit 120 is configured to receive image data obtained by the transducer of the probe 106 during an ultrasound scan. The image data refers to ultrasound data collected by the probe 106 while performing an ultrasound examination on a patient. For example, the image data may be collected during a fetal ultrasound and may therefore include various images of a patient's uterus and the fetal anatomy contained therein. The image processing circuit 120 may include multiple deep learning-based models configured to analyze the image data. For example, the image processing circuit may be configured to identify a view from which the image data is captured, an anatomical structure or other feature captured by the image data, the presence of a pathology in the image data, and so on. The image processing circuit 120 may be configured to identify the anatomical structure using one or more algorithms (e.g., image processing algorithms such as edge detection, machine learning models, deep neural networks, etc.). In some embodiments, the image processing circuit 120 may identify anatomical features such as bones, blood vessels, organs, etc., based on a shape, relative proximity, apparent depth, orientation, etc. of said features in the image data.

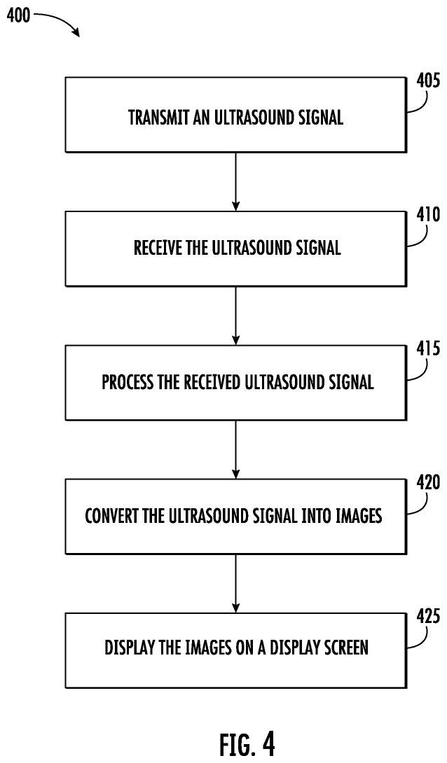

Referring to FIG. 4, a flow chart is shown illustrating a method 400 for performing an ultrasound imaging procedure using an ultrasound imaging system. In at least one embodiment, the ultrasound imaging system referred to by method 400 is the ultrasound imaging system 100 described above with reference to FIGS. 1-3, and method 400 may be implemented by the ultrasound imaging system 100. In some embodiments, method 400 may be implemented as executable instructions in a memory of the ultrasound imaging system 100, such as the memory 118 of FIG. 3.

At step 405, method 400 may include transmitting an ultrasound signal. The signal elements 108 (e.g., the transducer) of the probe 106 may transmit the ultrasound signal at step 405 by emitting high-frequency sound waves. For instance, when the transducer is brought into contact with a patient's body, the transducer sends the sound waves into the body. As the sound waves travel through different tissues and organs, they encounter boundaries between different types of tissues, such as muscle and bone. At these boundaries, some of the sound waves are reflected back to the transducer, while others continue to travel deeper into the body.

Then, at step 410, the transducer captures the sound waves that are reflected back from the tissue boundaries. These reflected waves, or echoes, are converted into electrical signals by the transducer. The strength and timing of these echoes provide information regarding the depth and density of the tissues they encountered at step 405 (which is used to create accurate images of the internal structures).

At step 415, the electrical signals are processed by ultrasound imaging system 100. In other words, once the ultrasound signals are received by the transducer and converted into electrical signals, the electrical signals undergo processing to enhance their quality and extract meaningful information. More specifically, the ultrasound imaging system 100 (e.g., the image processing circuit 120) may be configured to amplify the electrical signals and filter out noise. Advanced algorithms may be applied to analyze the echoes, determining the origin and intensity of the echoes. In some embodiments, step 415 may include adjusting parameters such as gain and depth to optimize the clarity of resulting images (e.g., displayed at step 425, as described below).

At step 420, the processed ultrasound signals are converted into ultrasound images. The ultrasound imaging system 100 is configured to translate the electrical signals into a visual format that represents the internal structures of the body. Specifically, step 420 may include mapping the intensity and location of the echoes to corresponding points on the ultrasound image.

The ultrasound images are displayed on a display screen (e.g., display device 132) at step 425 for real-time viewing. The ultrasound images may be displayed in grayscale, with varying shades representing different tissue densities. The ultrasound images can be adjusted for brightness, contrast, and other parameters to enhance visibility.

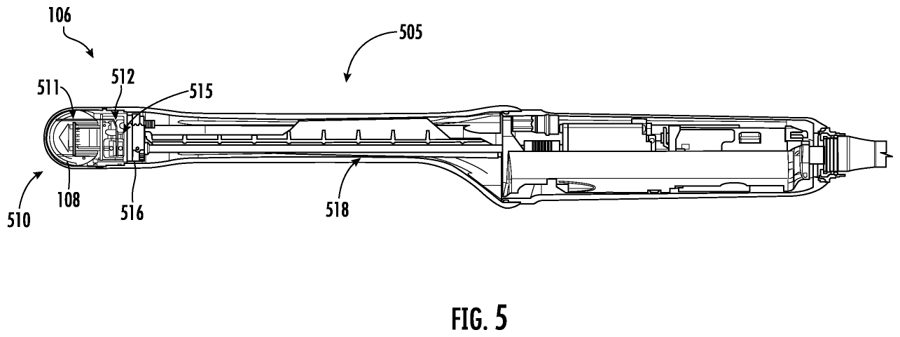

Referring to FIG. 5, the probe 106 is shown in greater detail. More specifically, the probe 106 shown in FIG. 5 may be an endocavity probe. The probe 106 is shown to include a distal portion 505 and a proximal portion 510 (e.g., a tip). The distal portion 505 extends away from the proximal portion 510. In some embodiments, the distal portion 505 is a handle of the probe 106. For example, a sonographer may hold the distal portion 505 during an ultrasound imaging procedure (e.g., a gynecological examination, an obstetric examination, a urological examination, etc.).

The probe 106 is also shown to include the signal elements 108. As described above, The signal elements 108 may be arranged in a transducer array. The transmit beamformer 102 and the transmitter 104 drive the signal elements 108 to emit pulsed ultrasonic signals into a body of a subject (e.g., a patient). For example, during a gynecological examination, a sonographer or other clinician may navigate the probe 106 proximate to a patient's uterus so that the signal elements 108 in the probe 106 emit the pulsed ultrasonic signals into the patient's uterus. The pulsed ultrasonic signals are then back-scattered from anatomical structures in the body, such as blood cells or muscular tissues, to produce echoes that return to the signal elements 108. That is, the signal elements 108 may include the transducer configured to transmit and receive the ultrasound signal, a matching layer configured to have an acoustic impedance match between a tissue to be imaged and a material of the transducer (e.g., such that the pulsed electronic signals can be back-scattered from the anatomical structures in the body and received as echoes by the signal elements 108), and a damping block configured to absorb ultrasound energy.

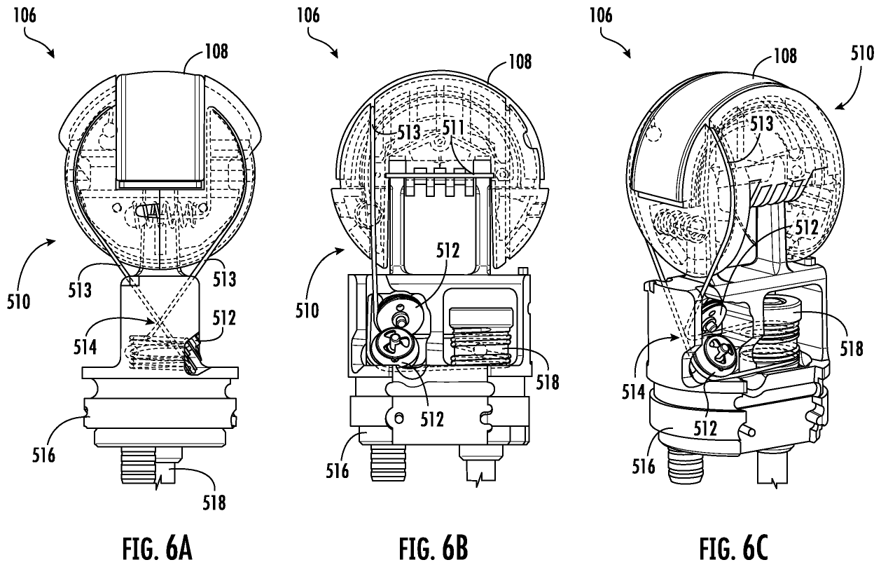

The proximal portion 510 may include a bearing 511, a pulley system including at least one pulley 512, and an oil volume 515. The bearing 511 is configured to facilitate rotational movement of the transducer. For example, according to the configuration of the probe 106 described herein, the bearing 511 may be configured to facilitate rotational movement of the transducer, allowing for a mechanical scanning range up to about 170 degrees (e.g., volume sweep angle). As another example, according to the configuration of the probe 106 described herein, the bearing 511 may be configured to facilitate rotational movement of the transducer, allowing for a mechanical scanning range corresponding to a field-of-view (FOV) of up to about 179 degrees. The at least one pulley 512 may be a mechanism by which the transducer is rotated (e.g., using a force imparted by drive mechanism 518), as described in greater detail below with reference to FIGS. 6A-6C. Furthermore, the proximal portion 510 includes an oil volume 515 configured to facilitate operation of the at least one pulley 512.

As shown in FIG. 5, the probe 106 may include a seal 516. The seal 516 may refer to a piece of hardware (e.g., a block, etc.) disposed between the distal portion 505 and the proximal portion 510. In this way, the seal 516 may be configured to prevent the oil volume 515 in the proximal portion 510 from reaching the distal portion 505. Advantageously, because signal elements 108 (e.g., transducer) configured to capture images are housed within the proximal portion 510 (e.g., and not the distal portion 505) of the probe 106, the oil volume 515 allows for ultrasound waves to propagate to and from the transducer across the proximal portion 510. Additionally, because mechanical components of the probe 106 (e.g., the at least one pulley 512) configured to rotate the transducer are housed within the proximal portion 510 (e.g., and not the distal portion 505) of the probe 106, the amount of oil required within the probe 106 is reduced such that the oil volume 515 is contained within the proximal portion 510, and not the distal portion 505.

As shown in FIG. 5, the probe 106 may include a drive mechanism 518. In some embodiments, the drive mechanism 518 may include a drive shaft. Furthermore, the drive mechanism 518 may include a motor. The drive shaft may be configured to transmit a force (e.g., rotational or linear motion) from the motor to the transducer (e.g., the signal elements 108). In turn, the force imparted on the transducer may cause the probe 106 to sweep an ultrasound beam across a region of interest (e.g., uterus, ovaries, prostate, rectal wall, etc.), thereby producing ultrasound images of the region of interest. In some embodiments, at least a portion of the drive mechanism 518 (e.g., a portion of the drive shaft, the motor, etc.) may be disposed within the distal portion 505 of the probe 106.

Referring to FIGS. 6A-6C, the proximal portion 510 of the probe 106 is shown in greater detail. Furthermore, FIGS. 6A-6C depict the at least one pulley 512 in greater detail. As shown, the at least one pulley 512 includes a cord 513 (e.g., a string, rope, etc.) that is configured to intersect (e.g., cross) at an intersection point 514. Due to the intersection point 514, the at least one pulley 512 (e.g., using a force provided by the drive mechanism 518) is configured to pull the cord 513 at an angle defined by the intersection point 514. In turn, the transducer may be rotated and angled up to 180 degrees from side-to-side (e.g., left-to-right). Furthermore, because the intersection point 514 of the cord 513 is disposed within the proximal portion 510 of the probe 106 (e.g., within the probe head), the probe 106 described herein may be configured to achieve a higher sweep angle compared to a conventional ultrasound probe (e.g., conventional ultrasound probes that include drive mechanisms, such as the drive mechanism 518, and pulleys, such as the pulley 512, in the handle (distal portion 505)). In some cases, the transducer can sweep up to 160 degrees from side-to-side (e.g., left-to-right) using the at least one pulley 512 described herein. In some cases, the transducer can sweep up to 170 degrees from side-to-side (e.g., left-to-right) using the at least one pulley 512 described herein.

In some embodiments, the transducer may be configured to pivot about a first axle within the proximal portion 510. In this way, the transducer may be configured to rotate about a first axis. The drive mechanism 518 may be configured to rotate about a second axis that is distinct from the first axis. As shown in FIGS. 6B and 6C, the at least one pulley 512 may include a first pulley 512 and a second pulley 512. As shown, the first pulley 512 and the second pulley 512 may be positioned within the proximal portion 510 such that each of the first pulley 512 and the second pulley 512 are configured to rotate about a distinct axis. Furthermore, the axes about which the first pulley 512 and the second pulley 512 are configured to rotate may be distinct from the first axis (e.g., about which the transducer is configured to rotate) and the second axis (e.g., about which the drive mechanism 518 is configured to rotate). In other words, the at least one pulley 512 in the proximal portion 510 may be positioned along an axis that is offset from the first axis and the second axis. It will be appreciated that the pulley system can include any number of pulleys to guide and control the cord 513 to ensure the cord 513 does not interfere with other components of the probe 106 (e.g., to ensure the cord 513 does not interface with a wall of a body of the probe 106 or cause friction forces during operation). For example, the pulley system could include, one pulley, two pulleys, three pulleys, four pulleys, five pulleys, 6 pulleys or more, with any number of the pulleys being located in the distal portion 505 of the probe 106 or in the proximal portion 510 of the probe 106. For example, in some embodiments, two or more pulleys 512 are located in at least one of the distal portion 505 of the probe 106 or in the proximal portion 510 of the probe 106.

As the drive mechanism 518 rotates about the second axis, the drive mechanism 518 may cause the cord 513 to wrap around a portion of the drive mechanism 518 (e.g., as shown in FIGS. 6B and 6C). Then, as shown, the cord 513 is configured to traverse the first pulley 512 and the second pulley 512. Using this configuration, by rotating the drive mechanism 518 in a first drive direction, the transducer is rotated in a first transducer direction. Similarly, by rotating the drive mechanism 518 in a second drive direction, the transducer is rotated in a second transducer direction. Such a configuration of the probe 106 provides a technical improvement over conventional ultrasound probes that may include the drive mechanism 518 and the pulley 512 in the handle (e.g., distal portion 505), such that the cord 513 is not pulled at an angle, as described herein.

Furthermore, as shown in FIG. 6B, the bearing 511 is disposed within the transducer, which allows the transducer (e.g., the signal elements 108) to extend around the spherical shape of the probe 106, thereby yielding a larger field of view for the probe 106 compared to conventional ultrasound probes in which the bearing 511 is disposed around the outside of the transducer.

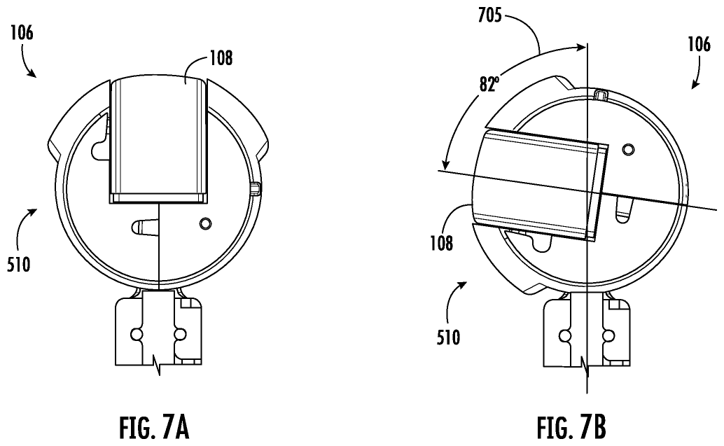

Referring to FIGS. 7A and 7B, the proximal portion 510 of the probe 106 is shown from an exterior perspective. As shown, the proximal portion 510 includes the signal elements 108, which extend along a portion of the exterior of the proximal portion 510 (e.g., the probe tip). FIG. 7B depicts an angle 705 by which the probe 106 (e.g., the transducer) is configured to rotate. As shown, a maximum amount of rotation of the transducer from a center position to one side (e.g., left or right). For instance, the angle 705 by the which the transducer is configured to rotate may be 82 degrees in a first direction (e.g., left, right, etc.). The angle 705 by which the transducer is configured to rotate is defined by an angle at the intersection point 514 of the cord 513, as described above with reference to FIGS. 6A-6C.

Referring to FIG. 8, a method 800 for using an ultrasound probe during an ultrasound imaging procedure is shown. In at least one embodiment, the ultrasound probe referred to by method 800 is the probe 106 described above with reference to FIGS. 5-7B, and method 800 may be implemented by the ultrasound imaging system 100. In some embodiments, method 800 may be implemented as executable instructions in a memory of the ultrasound imaging system 100, such as the memory 118 of FIG. 3. According to various example instances, the ultrasound imaging procedure referred to by method 800 may be a gynecological examination, an obstetric examination, or a urological examination.

As shown, at step 805, drive mechanism 518 may be used to transmit a force. In some embodiments, as described above, the force may be imparted to a proximal portion of the probe 106 via a drive shaft and using a motor. More specifically, the motor may be configured to generate a force (e.g., torque), which is transmitted through the drive mechanism 518 (e.g., via the drive shaft) to the proximal portion 510 (e.g., the probe head, tip, etc.). The drive shaft may be coupled to the motor and the proximal portion 510. That is, the drive shaft may be configured to direct a torque provided by the motor to the transducer (e.g., as described below with reference to step 820).

At step 810, the cord 513 is rotated around the drive mechanism 518. For instance, as shown in FIGS. 6B and 6C, the cord 513 may wrap around a portion (e.g., a tip, an end, a head, etc.) of the drive shaft. The cord 513 wraps around the drive mechanism 518 due to the force (e.g., torque) imparted by the motor. That is, the cord 513 may be positioned within the probe 106 such that when the cord 513 engages with the head of the drive shaft, the rotational motion is converted into tension along the cord 513.

Furthermore, the cord 513 may rotate around a first pulley 512 and a second pulley 512 at step 815. That is, once the cord 513 wraps around the drive mechanism 518, the cord 513 extends outward from the drive mechanism 518 and traverses the pulleys 512. Then, the pulleys 512 may be configured to guide the cord 513 along a defined path, maintaining tension and directional control. The cord 513 may be configured to cross over itself at intersection point 514 (e.g., as shown in FIGS. 6A and 6C).

At step 820, method 800 may include rotating the transducer using the drive mechanism 518 and the pulleys 512. That is, as the torque imparted on the drive mechanism 518 (e.g., rotational force imparted on the drive shaft from the motor) is converted to tension in the cord 513, the cord 513 is configured to provide impart a force configured to rotate the transducer. For instance, if the drive shaft rotates to the left, the transducer may be configured to rotate in a first direction responsive to the force imparted via the cord 513. Similarly, if the drive shaft rotates to the right, the transducer may be configured to rotate in a second direction responsive to the force imparted via the cord 513. As described in greater detail herein, the configuration of the pulleys 512 (e.g., along off-set axes and including the intersection point 514, as described above) within the proximal portion 510 yields a high sweep angle for the probe 106 and a larger field of view for the transducer compared to convention ultrasound probes.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As utilized herein, terms of degree such as “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to any precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that terms such as “exemplary,” “example,” and similar terms, as used herein to describe various embodiments, are intended to indicate that such embodiments are possible examples, representations, or illustrations of possible embodiments, and such terms are not intended to connote that such embodiments are necessarily extraordinary or superlative examples.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any element on its own or any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the drawings. It should be noted that the orientation of various elements may differ according to other exemplary embodiments, and that such variations are intended to be encompassed by the present disclosure.

As used herein, terms such as “engine” or “circuit” may include hardware and machine-readable media storing instructions thereon for configuring the hardware to execute the functions described herein. The engine or circuit may be embodied as one or more circuitry components including, but not limited to, processing circuitry, network interfaces, peripheral devices, input devices, output devices, sensors, etc. In some embodiments, the engine or circuit may take the form of one or more analog circuits, electronic circuits (e.g., integrated circuits (IC), discrete circuits, system on a chip (SOCs) circuits, etc.), telecommunication circuits, hybrid circuits, and any other type of circuit. In this regard, the engine or circuit may include any type of component for accomplishing or facilitating achievement of the operations described herein. For example, an engine or circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

An engine or circuit may be embodied as one or more processing circuits comprising one or more processors communicatively coupled to one or more memory or memory devices. In this regard, the one or more processors may execute instructions stored in the memory or may execute instructions otherwise accessible to the one or more processors. The one or more processors may be constructed in a manner sufficient to perform at least the operations described herein. In some embodiments, the one or more processors may be shared by multiple engines or circuits (e.g., engine A and engine B, or circuit A and circuit B, may comprise or otherwise share the same processor which, in some example embodiments, may execute instructions stored, or otherwise accessed, via different areas of memory).

Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be provided as one or more suitable processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given engine or circuit or components thereof may be disposed locally (e.g., as part of a local server, a local computing system, etc.) or remotely (e.g., as part of a remote server such as a cloud based server). To that end, engines or circuits as described herein may include components that are distributed across one or more locations.

An example system for providing the overall system or portions of the embodiments described herein might include one or more computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example embodiments described herein.

Although the drawings may show and the description may describe a specific order and composition of method steps, the order of such steps may differ from what is depicted and described. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions, and arrangement of the embodiments without departing from the scope of the present disclosure as expressed in the appended claims.

What is claimed is:

1. An ultrasound imaging system comprising:
a probe comprising:
a distal portion;
a proximal portion comprising:
a transducer configured to transmit and receive an ultrasound signal;
a bearing configured to facilitate rotational movement of the transducer; and
a pulley system comprising a pulley and a cord comprising an intersection point; and
a drive mechanism configured to impart a force on the transducer via the pulley system, the force applied at an angle defined by the intersection point of the cord.

2. The ultrasound imaging system of claim 1, wherein the transducer is configured to rotate about a first axis, and the drive mechanism is configured to rotate about a second axis.

3. The ultrasound imaging system of claim 2, wherein the pulley is positioned along a third axis, and the third axis is offset from the first axis and the second axis.

4. The ultrasound imaging system of claim 1, wherein the probe is an endocavity probe, and the distal portion is a handle of the endocavity probe.

5. The ultrasound imaging system of claim 1, wherein the probe further comprises a seal disposed between the distal portion and the proximal portion.

6. The ultrasound imaging system of claim 5, wherein the proximal portion further comprises an oil volume, and the seal is configured to prevent the oil volume from reaching the distal portion.

7. The ultrasound imaging system of claim 1, wherein the pulley is a first pulley, and the pulley system comprises the first pulley and a second pulley.

8. The ultrasound imaging system of claim 1, wherein the bearing is configured to facilitate rotation of the transducer up to 180 degrees.

9. An ultrasound probe comprising:
a distal portion;
a proximal portion comprising:
a transducer configured to transmit and receive an ultrasound signal;
a bearing configured to facilitate rotational movement of the transducer; and
a pulley system comprising a pulley and a cord having an intersection point; and
a drive mechanism configured to impart a force on the transducer via the pulley system, the force applied at an angle defined by the intersection point of the cord.

10. The ultrasound probe of claim 9, wherein the transducer is configured to rotate about a first axis, and the drive mechanism is configured to rotate about a second axis.

11. The ultrasound probe of claim 10, wherein the pulley is positioned along a third axis, and the third axis is offset from the first axis and the second axis.

12. The ultrasound probe of claim 9, wherein the ultrasound probe is an endocavity probe, and the distal portion is a handle of the endocavity probe.

13. The ultrasound probe of claim 9, wherein the ultrasound probe further comprises a seal disposed between the distal portion and the proximal portion.

14. The ultrasound probe of claim 13, wherein the proximal portion further comprises an oil volume, and the seal is configured to prevent the oil volume from reaching the distal portion.

15. The ultrasound probe of claim 9, wherein the pulley is a first pulley, and the pulley system comprises the first pulley and a second pulley.

16. The ultrasound probe of claim 9, wherein the bearing is configured to facilitate rotation of the transducer up to 180 degrees.

17. An ultrasound imaging system comprising:
a probe comprising:
a distal portion;
a proximal portion comprising:
a transducer configured to transmit and receive an ultrasound signal;
a bearing configured to facilitate rotational movement of the transducer; and
a pulley system comprising a pulley and a cord having an intersection point;
a seal disposed between the distal portion and the proximal portion, wherein the seal is configured to prevent an oil volume within the proximal portion from reaching the distal portion; and
a drive mechanism configured to impart a force on the transducer via the pulley, the force applied at an angle defined by the intersection point of the cord.

18. The ultrasound imaging system of claim 17, wherein the transducer is configured to rotate about a first axis, the drive mechanism is configured to rotate about a second axis, and the pulley is positioned along a third axis, wherein the third axis is offset from the first axis and the second axis.

19. The ultrasound imaging system of claim 17, wherein the probe is an endocavity probe, and the distal portion is a handle of the endocavity probe.

20. The ultrasound imaging system of claim 17, wherein the pulley is a first pulley, and the pulley system comprises the first pulley and a second pulley.
